(19) United States
(12) Patent Application Publication
(10) Pub. No.: US 2016/0287430 A1
(43) Pub. Date: Oct. 6, 2016

(54) Condom

(71) Applicant: Aretha Joy Thomas, Chatham, VA (US)

(72) Inventor: Aretha Joy Thomas, Chatham, VA (US)

(21) Appl. No.: 14/672,458

(22) Filed: Mar. 30, 2015

(51) Int. Cl. A61F 6/04

(52) U.S. Cl. CPC A61F 6/04; A61F 2006/047

(57) ABSTRACT

A condom used by males during sexual intercourse. The first portion includes a semen catch that goes over the tip of the penis. The second portion includes a glans tip cover that goes over the glans. The semen catch spirals down on top of the glans tip cover and stays in place over the glans until ejaculation, wherein the semen catch unfurls. The condom may include a strong comfortable corona ring that goes snuggly around the corona and rests on the circumcision scar.

### BACKGROUND OF THE INVENTION

1. Field of the Invention

The present disclosure is generally directed to sheath devices designed to protect against venereal infection during sexual intercourse and pregnancy and, more particularly, toward condom or sheath devices providing protection for the glans area, as well as having an improved fit and/or increased sensation and/or increased pleasure and/or enhanced erectile performance.

2. Background of the Invention

The present invention is directed to a condom for contraceptive and prophylactic use, and ultimately, enhanced pleasure. This invention relates to an improved embodiment for vaginal and anal intercourse which encourages its use beyond merely for prevention and protection. HIV and other STDs continue to ravage the world. Lives continue to be lost and the costs of treating those infected have the potential of destabilizing states and nations.

Dramatically unplanned pregnancies also have negative effects of society and the global population. Common issues that people report while using traditional condoms are the lack of sensation, the loss of spontaneity while opening and applying the condom after erection and before intercourse begins, and the difficulty of maintaining an erection while the condom is on. Aside from these frequency stated frustrations, there is a reality that traditional condoms are able to slide off during intercourse. Typically the condom will slide off during intercourse, leaving the partner unprotected.

The present disclosure is directed toward overcoming one or more of the above-identified problems.

### SUMMARY OF THE INVENTION

In view of the foregoing, it is an object of the present invention to provide a condom that is safer than currently available commercial condoms.

It is an object of the present invention to provide an improved condom for use in receptive intercourse.

It is an object of the present invention to provide an improved condom that can be easily inserted for receptive intercourse.

It is an object of the present invention to provide an improved condom that can be applied before intercourse so as not to interrupt sexual activity.

It is an object of the present invention to provide an improved condom that does not slip during intercourse.

It is an object of the present invention to provide an improved condom that can loosely accommodate a penis so as not to cause loss of sensation.

It is an object of the present invention to provide an improved condom that can enhance direct tactile contact and friction between the penis of the internal condom wall during sexual or anal intercourse.

It is an object of the present invention to provide an improved condom that can readily stretch beyond one size to accommodate penises of different sizes, from smaller to extra-large.

A condom device in accordance with the present invention is disclosed. The condom device includes a glans tip cover that covers the head of the penis with a semen catch that is attached to the glans tip to catch semen at time of ejaculation. The semen catch is very thin and comfortable not taking away the sensation during intercourse.

In one embodiment, the corona ring, located at the end of the glans tip cover, goes over the corona and stops at the circumcision scar, wherein the corona ring has an elasticity enabling it to fit snuggly around the circumcision scar.

In one embodiment, the middle portion of the embodiment includes a top shaft strap that goes along the top of the penis. In one example, the thickness of the middle portions includes a tubular strap that is elongated from the Corona ring. The top shaft strap elongates to the top of the base ring.

In one embodiment, the middle portion of the embodiment includes a bottom shaft strap that goes along the bottom of the penis. In one example, the thickness of the middle portions includes a tubular strap that is elongated from the Corona ring. The bottom shaft strap elongates to the bottom part of the base ring.

In another embodiment, a base ring is attached to the elongated top and bottom shaft straps that end at the base of the penis, holding the shaft straps in place. The base ring is a durable thick ring that has elasticity for comfort. The base ring is attached to a testicle hold ring.

In another embodiment, the testicle hold ring is a thick tubular ring that goes around the scrotum to hold the base ring in place to avoid slipping. The embodiment is attached to the bottom of the base ring and has elasticity for comfort.

In yet further embodiments, the condom device may be made of polyurethane, nitrile, latex or other man-made materials.

In still a further embodiment, the thickness of the middle, end and bottom portions are thicker than the top portion for durability.

When the condom is placed over the male genitalia, the middle portion is separated with the tubular straps and fits the portions of the body between the glans and the scrotum.

A device in accordance with an exemplary embodiment of the present invention is disclosed. The condom device includes a portion to cover the glans, as well as a corona ring that fits snuggly on the circumcision scar. The tubular portion has a thickness and durability goes along the elongated part of the penis, and the base ring portion of the embodiment holds the tubular portion in place. When the condom is on a user, the base ring is positioned around the base of the male genitalia area and the testicle hold ring goes around the scrotum.

It is an object of the present disclosure to provide an improved condom device that protects against most STDs, while providing sexual satisfaction to the wearer in terms of improved sensation, increase spontaneity, and enhanced erectile stimulation.

Other objects, aspects and advantages of the present disclosure can be obtained from a study of the specification, the drawings and the appended claims.

### BRIEF DESCRIPTION OF DRAWINGS

FIG. 1 is a top view of the first embodiment of the condom device in accordance with the present disclosure;

FIG. 2 is an isometric side view of the first embodiment of the condom device in accordance with the present disclosure;

FIG. 3 is a perspective front view of the first embodiment of the condom device in accordance with the present disclosure;

FIG. 4 is an isometric view of the first embodiment of the condom device in accordance with the present disclosure;

FIG. 4A is a perspective broad view of the first embodiment of the condom device in accordance with the present disclosure;

FIG. 5 is a broad view of the second embodiment of the condom device in accordance with the present disclosure;

FIG. 6 is a perspective view of the second embodiment of the condom device in accordance with the present disclosure;

FIG. 7 is a perspective top view of the second embodiment device in accordance with the present disclosure;

FIG. 8 is a perspective side view of the second embodiment of device in accordance with the present disclosure;

FIG. 9 is an isometric view of second embodiment of device in accordance with the present disclosure;

FIG. 10 is a perspective view of third embodiment of device in accordance with the present disclosure;

FIG. 11 is a dimensional front view of third embodiment of device in accordance with the present disclosure;

FIG. 12 is an isometric view of third embodiment of device in accordance with the present disclosure;

FIG. 13 is a bottom view of third embodiment of device in accordance with the present disclosure;

FIG. 14 is a broad view of forth embodiment of device in accordance with the present disclosure;

FIG. 15 is a perspective view of forth embodiment of device in accordance with the present disclosure;

FIG. 16 is an isometric detailed view of forth embodiment of device in accordance with the present disclosure;

FIG. 17 is a partial isometric view of the forth embodiment of device in accordance with the present disclosure;

FIG. 18 is a partial view of the fifth embodiment of device in accordance with the present disclosure;

FIG. 19 is a perspective view of the fifth embodiment of device in accordance with the present disclosure;

FIG. 20 is an isometric front view of the fifth embodiment of device in accordance with the present disclosure;

FIG. 21 is a perspective horizontal view of the fifth embodiment of device in accordance with the present disclosure;

FIG. 22 is a vertical view of the fifth embodiment of the device in accordance with the present disclosure;

FIG. 23 is a dimensional view of the sixth embodiment of the device in accordance with the present disclosure;

FIG. 24 is a perspective bottom view of the sixth embodiment of the device in accordance with the present disclosure;

FIG. 25 is an isometric detailed view of the sixth embodiment of the device in accordance with the present disclosure;

FIG. 26 is a perspective view of the sixth embodiment of the device in accordance with the present disclosure;

FIG. 27 is an alternative side view of the sixth embodiment of the device in accordance with the present disclosure;

FIG. 28 is a perspective view of the seventh embodiment of the device in accordance with the present disclosure;

FIG. 29 is an alternative top view of the seventh embodiment of the device in accordance with the present disclosure;

FIG. 30 is a front view of the seventh embodiment of the device in accordance with the present disclosure;

FIG. 31 is a perspective side view of the embodiment of the device in accordance with the present disclosure;

FIG. 32 is a side view of the whole embodiment of the device in accordance with the present disclosure;

FIG. 33 is a perspective top view of embodiment of the device in accordance with the present disclosure; and

FIG. 34 is a perspective back view of embodiment of the device in accordance with the present disclosure.

### DETAILED DESCRIPTION OF THE INVENTION

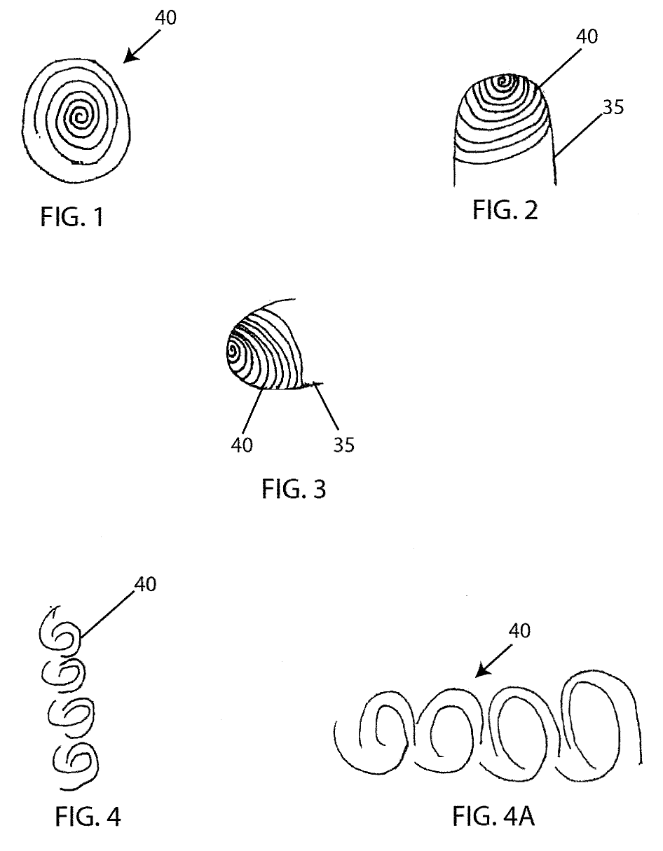

In the following description, it will be understood that the illustrations referred to are for the purpose of describing embodiments of the invention and are not intended as limitations. FIG. 1 illustrates a perspective top view of one embodiment of a semen catch 40 for a condom that is spiral and attached to the glans tip cover 35 and adapted to fit over the glans/tip/head of a penis. FIGS. 2 and 3 shows the glans tip cover 35 in its attached state. In the attached state, the glans tip cover 35 remains in a spiral configuration. Glans tip cover 35 remains in the attached state until a user ejaculates during intercourse. FIGS. 4 and 4A show the semen catch 40 in its extended form after ejaculation takes place, allowing the semen catch to unfurl as the semen discharges.

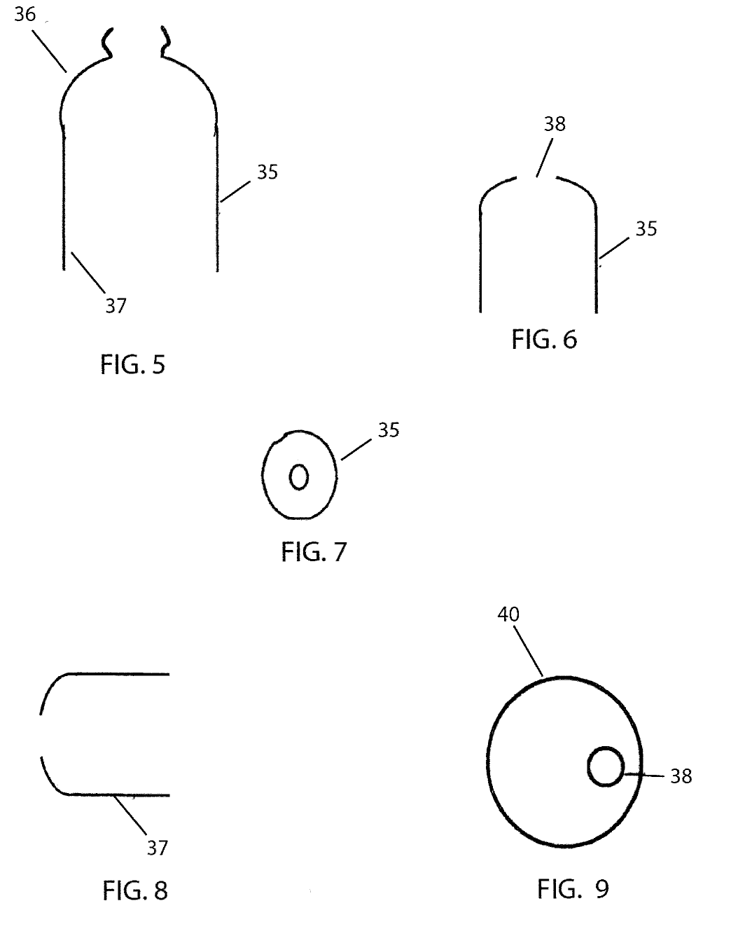

FIG. 5 shows another embodiment wherein the condom includes a glans tip cover 35 adapted to fit over most glans and doesn't require rolling for use. The glans tip cover 35, which is typically made from a thin, elastic latex or plastic, has a generally circular tubular body 36 having a first open end 37 is made of thin elastic latex. FIG. 6 shows an embodiment where dome top 38 (which is not an open end) has the traditional tip like a globe, but also has a semen catch that is attached to the end (not shown). FIG. 7 is another view of the glans tip cover to show additional details. FIG. 9 illustrates a view of the opening of the attached semen catch 40 in combination with the dome top 38. In the embodiments depicted in FIG. 5-9, the semen catch 40 comes over the glans tip cover in the form of a spiral around the glans tip to such time of ejaculation.

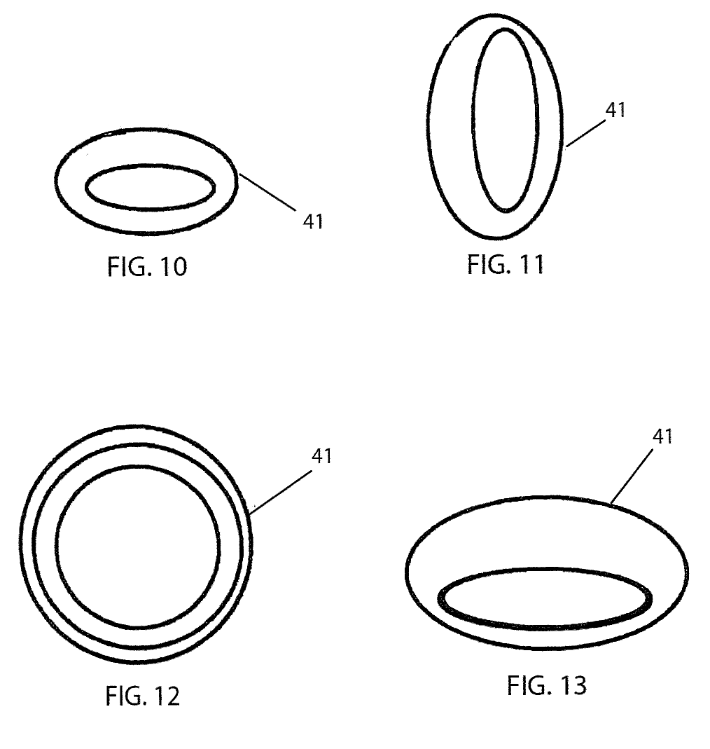

The corona ring 41, as shown in FIGS. 10-13, is a thick elastic ring that is adapted to fit on the circumcision scar after the corona. The corona ring 41 adds protection to the glans cover and secures the glans tip in place. The corona ring 41 at FIG. 11 is a view of one position; it is evenly shape in a circle and elastic for comfort yet having durability and strength.

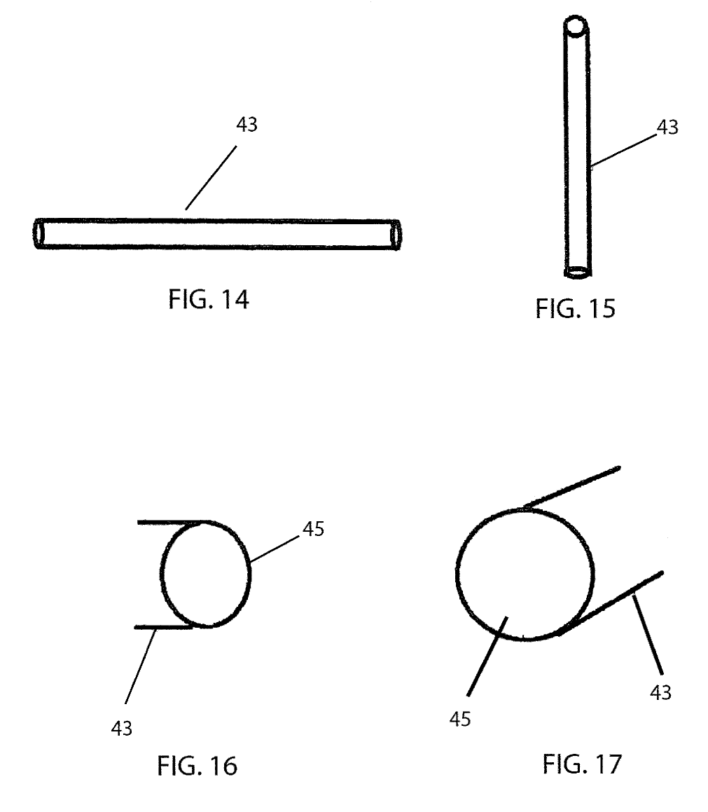

FIGS. 14-17 shows a top shaft strap 43 for a condom in accordance with another embodiment. The shaft strap 43 is a straight tubular strap with elasticity for comfort and durability. Top shaft strap 43 may vary in size in other embodiments. The embodiment as shown in FIG. 16 is an isometric view of the inside of the shaft strap. As seen in FIG. 16, the inside of the shaft strap 45 is not hollow. FIG. 17 is a perspective view of the embodiment.

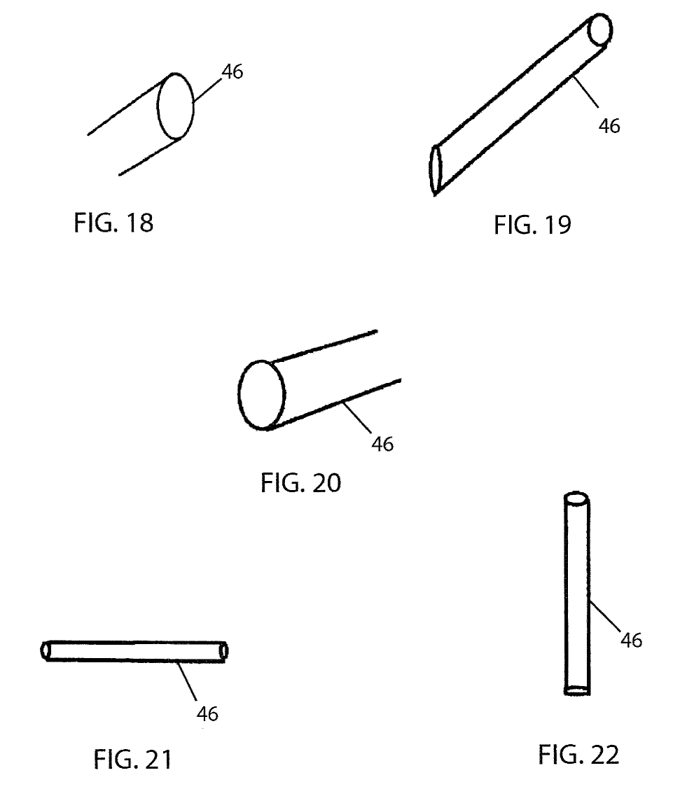

FIGS. 18-22 shows the bottom shaft strap. Bottom shaft strap 46 is located on the bottom of said condom. The shaft strap 46 comprises a tubular end that is connected to the corona ring and another end that is connected to a base ring. In one embodiment, bottom shaft strap 46 may be an elastic tubular strap that can vary in length to accommodate the need of the male using said condom.

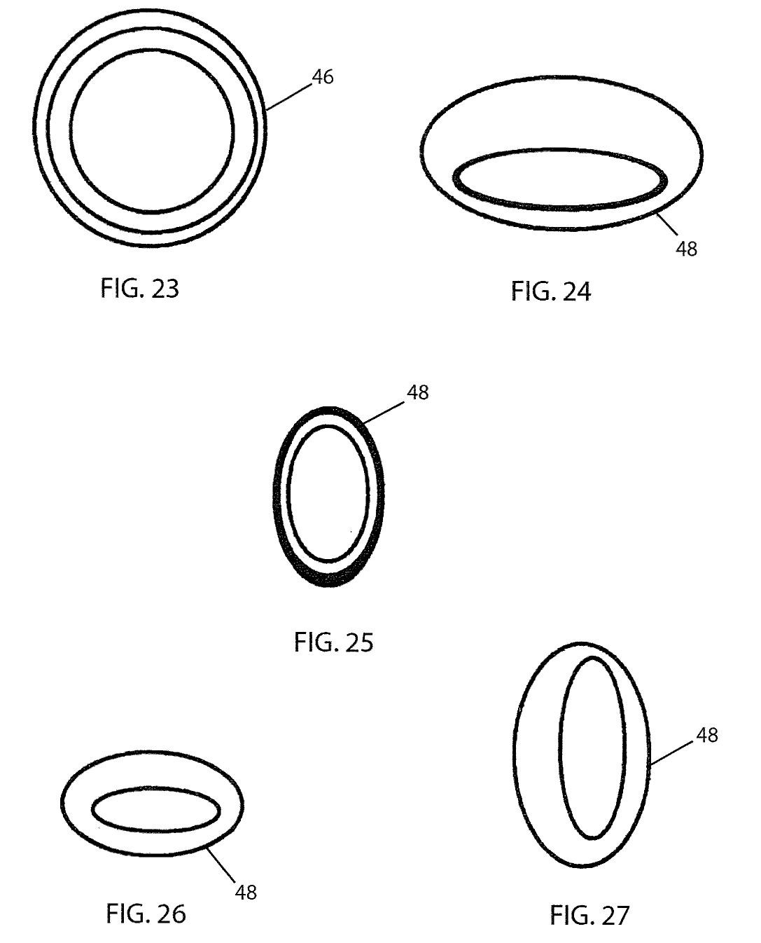

FIGS. 23-27 depict the base ring 48, which comprises a tubular ring adapted to be placed around the base of the penis. The base ring 48 is attached to the top shaft strap 43 and comprises a perfectly round ring with elasticity for comfort.

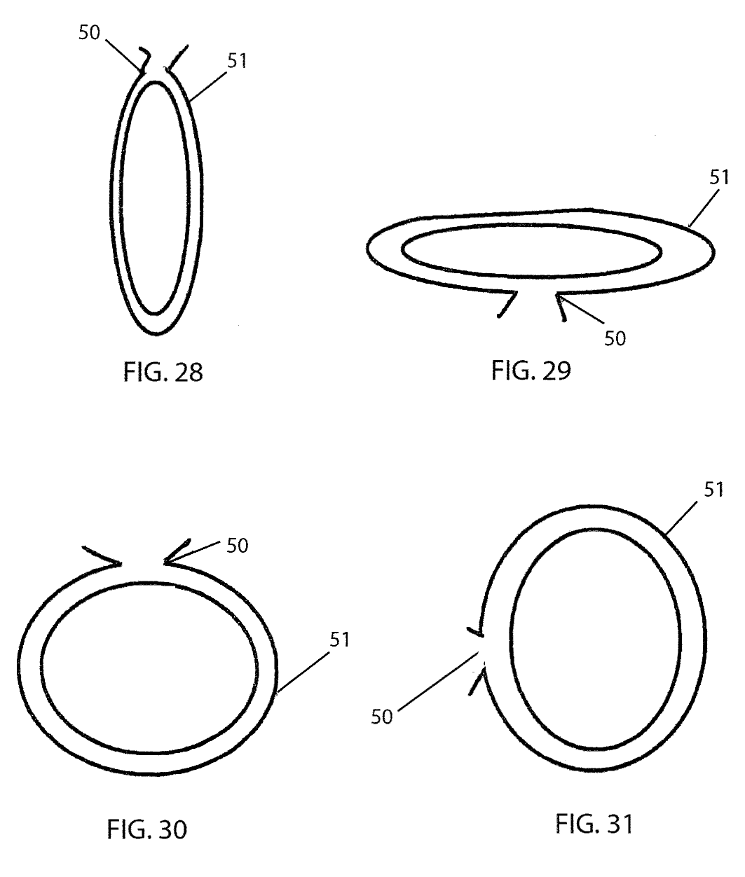

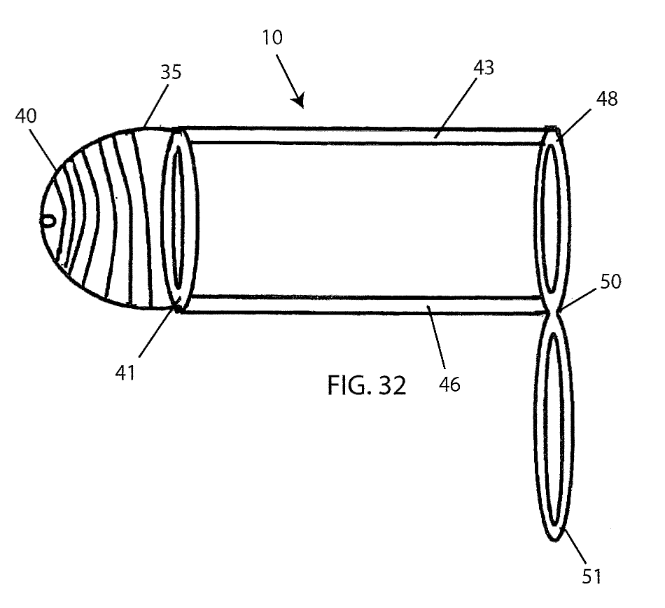

FIGS. 28-31 show an enlarged view of testicle hold ring 51. The testicle hold ring 51 attaches to the base ring 48 at attachment 50. The testicle hold ring 51 has elasticity and is slightly bigger in size to accommodate the scrotum. The elasticity will add comfort once wrapped around the male scrotum.

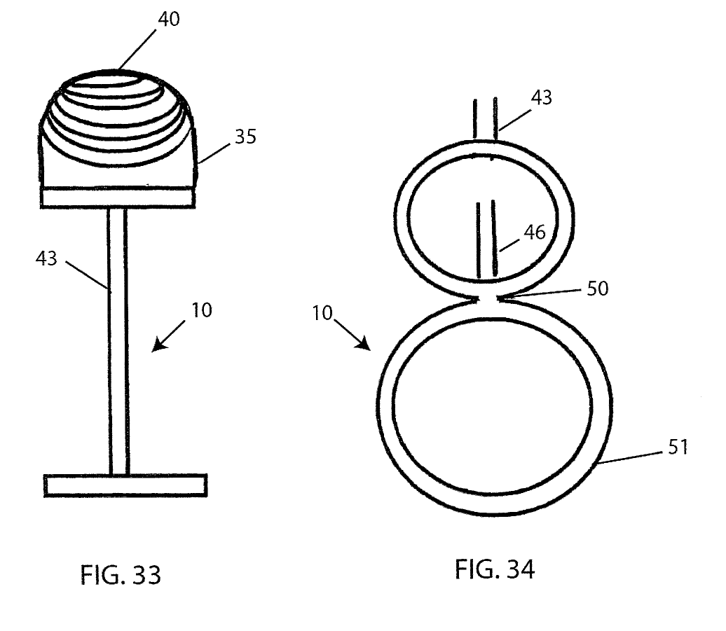

FIGS. 32-34 depicts an embodiment of condom 10 in its entirety. As shown in FIGS. 32 and 33, the semen catch 40 is attached to the glans tip cover 35, shown in its attached state before ejaculation occurs. The condom 10 includes the attachment 50 to the base ring 48 and testicle ring hold 51 which causes the condom to stay in place. FIG. 32 shows the top and bottom shaft straps 43, 46 that are connected to the corona ring 41 as well as the base ring 48 to hold the corona ring 41 on the circumcision scar in place thus preventing the condom from moving or slipping off.

FIG. 33 is a perspective top view of the embodiment with the semen catch 40 and glans tip cover 35 in its natural attached state. The top and bottom shafts straps 43, 46 are perfectly in line with each other; this holds the condom in place and prevents slipping.

FIG. 34 shows the back of the condom comprising the base ring 48 and the testicle hold ring 51 that goes over the base and scrotum, respectively, and are held together with a thick elasticity that will allow comfort as it is wrapped around both the base and scrotum. As shown, the testicle hold ring is slightly larger than the corona ring as well as the base ring for comfort when placed on scrotum.

The condom should be made of a flexible non-permeable material such as, for example, latex, polyurethane, nitrile, etc. The condom should be a reservoir tip where the semen can collect. The condom should have a portion to accommodate the scrotum.

With respect to the above description, it is understood that the optimum dimensional relationships and physical properties for the parts of the inventive condom 10 to include variations in for example, size materials, shape, form, thickness, shore hardness, tensile, strength, elongation modules, flexural modulus, specific gravity, function and manner of operations, assembly and use. These variations are deemed readily apparent and obvious to one skilled in the art, and all equivalent relationships to those illustrated to the drawings and described in the specifications, are intended to be encompassed by the present invention.

By making condoms more enjoyable and user friendly, the present invention will increase condom usage and in turn, help reduce the spread of STD's. The inventive condom will also increase protection HIV/AIDS and most STDs.

Additionally, one skilled in the art will appreciate that many variations of the condom device of the present invention are possible without departing from the spirit and scope of the present invention. These variations can include, but are not limited to, the following variations which can be implemented singularity or in combination:

1) Different materials for the condom such as, for example, latex, polyisoprene, polyurethane, etc. or any combination thereof.

2) Various lengths and widths of the condom, as well as various sizes of the scrotum covering portion.

3) Various sizes of the condom.

4) Various configurations of the condom as shown, but not limited to, the attached drawings, which are to be considered exemplary only.

5) Various textures in on the outside that can enhance the pleasure.

6) Various lubricants use with the condom such as, but not limited to, water-based lubricants, microbicides gels, flavored lubricants, heating or cooling sensation lubricants, etc.

It will be apparent to those skilled in the art of numerous modifications and variations of the described examples and the embodiments are possible in the light of the above teachings of the disclosure. The disclosed example and embodiments are presented for purposes of illustration only. Other alternate embodiments may include some or all of the features disclosed herein. Therefore, it is the intent to cover all such modifications and alternate embodiments as may come within the true scope of this invention, which is to be given in full breadth thereof. Additionally, the disclosure of a range of values is a disclosure of every numerical value within that range.

1-16. (canceled)

17. A condom comprising:
a glans tip cover having a thin walled portion and adapted to cover the glans of a penis, said glans tip cover including a semen catch having an attached state and an elongated state, said semen catch at least partially covering and overlapping said glans tip cover when in said attached state, wherein said semen catch is adapted to cover a portion of the glans when in said attached state and unfurls when in said elongated state;
a corona ring attached to an end of the glans tip cover opposite of the semen catch and adapted to go over the corona of a penis and fit at the end of the glans tip stopping at the circumcision scar;
a base ring adapted to fit around the base of the penis;
a top shaft strap attached at one end to a portion of the corona ring and attached at an opposing end to a portion of the base ring;
a bottom shaft strap attached at one end to a portion of the corona ring and attached at an opposing end to a portion of the base ring; and
a scrotum ring tangentially connected to the base ring and adapted to fit around the scrotum to further secure the position of the base ring,
wherein the base ring holds the top and bottom shaft straps in place and prevents the corona ring from sliding off.

18. The condom of claim 17 wherein the semen catch comprises a tubular closed-end body with a spiral component adapted to elongate at the time of ejaculation.

19. The condom or claim 17 wherein the glans tip cover is adapted to elongate to a length fitting over the glans tip and stopping at the corona of a penis.

20. The condom of claim 17 wherein the corona ring is an elastic ring having an elasticity adapted to fit on the circumcision scar of the penis keeping the glans tip cover in place.

21. The condom of claim 17 wherein the top shaft strap and the bottom shaft strap are comprised of an elastic material.

22. The condom of claim 21 wherein the elastic material is selected from the group consisting of

23. The condom of claim 17 wherein the top and bottom shaft straps are a tubular elastic that go up the shaft of the penis to the base ring.

24. The condom of claim 17 wherein the corona ring is an elastic tubular ring.

25. The condom of claim 17 wherein the base ring is an elastic tubular ring.

26. The condom of claim 17 wherein the scrotum ring is an elastic tubular ring.
